(19) United States
(12) Patent Application Publication
(10) Pub. No.: US 2015/0191695 A1
(43) Pub. Date: Jul. 9, 2015

(54) COMPOSITION INCLUDING GELSOLIN AS EFFECTIVE INGREDIENT FOR INDUCING DIFFERENTIATION INTO DENDRITIC CELL AND METHOD OF INDUCING DIFFERENTIATION INTO DENDRITIC CELL

(71) Applicant: KOREA INSTITUTE OF RADIOLOGICAL & MEDICAL SERVICES, Seoul (KR)

(72) Inventors: Jie Young Song, Goyang-si (KR); Mi Hyoung Kim, Seoul (KR); Ji Yeon Ahn, Seoul (KR); In Sung Jung, Seoul (KR); Dae Seog Lim, Seongnam-si (KR)

(21) Appl. No.: 14/591,012

(22) Filed: Jan. 7, 2015

(30) Foreign Application Priority Data
Jan. 8, 2014 (KR) 10-2014-0002282

(51) Int. Cl. C12N 5/0784; A61K 39/00

(52) U.S. Cl. CPC C12N 5/0639; A61K 39/0011; C12N 2501/998; C12N 2506/11; A61K 2039/5154

(57) ABSTRACT

Disclosed are a composition including gelsolin as an effective ingredient for inducing differentiation into a dendritic cell, a method of inducing differentiation by using the composition, and a vaccine composition for treating cancer or an immune-mediated disease. In addition, a composition including gelsolin as an effective ingredient for inducing differentiation into a dendritic cell and a method of inducing differentiation into a dendritic cell by treating an immature dendritic cell with gelsolin are also disclosed. Further, a dendritic cell differentiated by using the method of inducing differentiation into the dendritic cell and a vaccine composition including the dendritic cell as an effective ingredient for treating cancer or an immune-mediated disease are disclosed.

### CROSS-REFERENCE TO RELATED PATENT APPLICATION

This application claims the benefit of Korean Patent Application No. 10-2014-0002282 filed on Jan. 8, 2014, in the Korean Intellectual Property Office, the disclosure of which is incorporated herein in its entirety by reference.

### BACKGROUND

The present disclosure relates to a composition including gelsolin as an effective ingredient for inducing differentiation into a dendritic cell, a method of inducing differentiation into the dendritic cell, and a vaccine composition for treating an immune disease by using the method.

Dendritic cells first discovered from the skin by Langerhans in 1868 are known to play an important role in induction and regulation of immunity as professional antigen-presenting cells (APCs) in the 1990s. The dendritic cells are present in trace amounts in human body, but consist of a heterogeneous group having a distinct phenotype from macrophages. The dendritic cells are capable of inducing a primary immune response that can stimulate naive T cells, which have not been exposed to antigens. In addition, the dendritic cells are the immune cells having characteristics capable of inducing an immunological memory. The dendritic cells are capable of performing a strong immune activation since the dendritic cells, as APCs, express MHC molecules (I/II), co-stimulatory molecules, such as CD80 and CD86, and adhesion molecules, such as ICAM-1, on a cell surface at high concentration and secret various cytokines (e.g., IFN-alpha, IL-12, IL-18, or the like).

Differentiation into the dendritic cells occurs in an ex vivo culture manner by using monocytes derived from peripheral blood or hematopoietic stem cells derived from peripheral blood, cord blood, and bone-marrow, and the ex vivo culture consists of two steps. The first step is to induce differentiation of precursor cells, such as monocytes and hematopoietic stem cells, to immature dendritic cells in terms of phenotype and function thereof; and the second step is to complete differentiation of the immature dendritic cells to mature dendritic cells by using a differentiating material for maturation in terms of phenotype and function of the cells depending on the application. In particular, the phenotype and the function of the dendritic cells that are differentiated in the second step can be regulated by using the differentiating material, and accordingly, aspects of immunity enhancement or immunity inhibition are clearly distinguishable. In this regard, differentiation into the dendritic cells needs to be appropriately regulated in vitro to satisfy the best conditions required for a variety of immune disorders and symptoms. Research regarding the needs above has been actively progressed.

Methods of preparing dendritic cells have been suggested so far, to enhance cellular immunity by using various differentiation-inducing factors. Here, examples of the differentiation-inducing factors are cytokine cocktail, which is a combination of various cytokines, pathogen-associated molecular patterns (PAMPs) that are commonly shared by infectious factors, toll-like receptor ligands recognizing the PAMPs, toxic-free sterilized bacteria and apoptosis-inducing cancer cells. In particular, in order to induce maturation of the immature dendritic cells that are artificially prepared in vitro, bacterial-derived or viral-derived materials, such as LPS, poly(I:C), LTA, CpG, or flagellin, or synthetic molecules corresponding thereto may be used alone or in various combinations. In most cases of immune diseases, balance between Th1 and Th2 cells is important. In the case of cancer, asthma, or atopy where Th2 cells are activated, the secretion of IL-12 from the dendritic cells is necessary to operate Th1 cells.

Based on these details, various methods have been used to induce the maturation of the dendritic cells. These dendritic cells are found to have an increase in immunological activity by in vitro analysis while the cells have unsatisfactory results in clinical studies targeting on the human body. These results may come from that the production of IL-12, which is essential to the induction of cellular immunity, is very low in the dendritic cells prepared in vitro, the viability and mobility of the dendritic cells are low when administered into the body, and the dendritic cells lose functions thereof upon systemic immunosuppressive environment. Therefore, the development of techniques that can efficiently enhance immune activity of Th1 cells in the body is demanded by the enhancement of function, mobility, and viability of the dendritic cells.

Gelsolin is an 84-kD actin-binding protein that is known to exist in cytoplasm, and is also involved in remodeling of actin fibers that are necessary for making cell shape and conducting cell movement. In this regard, cells of a gelsolin-deficient mouse are reported to have reduction in mobility and defects in cytoskeleton. In gelsolin-deficient fiber cells, actin fibrosis has been excessively occurred due to a lack of appropriate cutting and remodeling of the actin fibers. Although roles of gelsolin secreted to the outside the cell are not well known, gelsolin is expected to have a role of removing F-actin that is secreted during apoptosis.

Gelsolin, which is a normal serum protein, is divided into a cytoplasmic protein and a secretory protein derived by alternative splicing of a single gene, and gelsolin has a similar structure in both divided forms. Secretory gelsolin is called plasma gelsolin (pGSN) and is present in blood of humans and rodents in a concentration of 250±50 ug/ml. Many of functions and roles of gelsolin (pGSN) are not known, but in clinical studies, it is reported that an amount of gelsolin is reduced in inflammation and sepsis. Studies of gelsolin on immune cells are not substantially reported yet in addition to the findings in the clinical. According to the present invention, gelsolin is found to act as a differentiation-inducing factor that is commonly used in differentiation of the immature dendritic cells, and in this regard, immunological functions of the mature dendritic cells as cancer vaccines using gelsolin are proposed.

The inventors of the present invention provides a method of inducing differentiation to mature dendritic cells by using gelsolin and an application of the dendritic cells as a therapeutic agent for an immune disease based on effects of the dendritic cells on antitumor and immune-potentiating activity. In greater detail, when gelsolin is treated with immature dendritic cells, the immature dendritic cells may be sufficiently differentiated to mature dendritic cells, which can then show capability of antigen presentation necessary to activate T cells. In addition, the mature dendritic cell shows a significant increase in capability of producing IL-12, which is an essential factor for forming immune environment of Th1 cells. Accordingly, use of a gelsolin-treated dendritic cell as a vaccine for treating cancer or other immune-mediated diseases such as asthma or atopy is confirmed, thereby completing the present invention.

### PRIOR ART DOCUMENT

### Patent Document

KR 10-2009-0004966 (published on Jan. 12 2009)

### SUMMARY

Provided is a composition including gelsolin as an effective ingredient for inducing differentiation to a dendritic cell.

Provided is a method of inducing differentiation to a dendritic cell by treating an immature dendritic cell with gelsolin.

Provided are a dendritic cell differentiated according to the differentiation induction method and a vaccine composition including the dendritic cell as an effective ingredient for treating cancer or other immune-mediated diseases.

Additional aspects will be set forth in part in the description which follows and, in part, will be apparent from the description, or may be learned by practice of the presented exemplary embodiments.

In order to achieve objects above, there is provided a composition including gelsolin as an effective ingredient for inducing differentiation to a dendritic cell.

In detail, the dendritic cell has positive immunological characteristics with respect to at least one surface antigen selected from the group consisting of CD40, CD80, CD86, and MHC class II molecules.

The term “dendritic cell” as used herein is a professional antigen-presenting cell (APC) and has capability of inducing a primary immune response that can stimulate a naive T cell, which has not been exposed to an antigen, and capability of inducing an immunological memory. A mature dendritic cell can provide all the signals required for T cell activation and proliferation, and in this regard, a specific marker that is expressed in the mature dendritic cell may be used to identify types and differentiation of dendritic cells. Accordingly, the dendritic cell can be specified with respect to humans and mammals other than humans.

The term “gelsolin” as used herein refers to wild-type gelsolin (GenBank accession No: X04412), isoforms, analogs, variants, fragments or functional derivatives of gelsolin. Gelsolin also includes pure, synthetic, and recombinant gelsolin and a derivative of gelsolin. Gelsolin, specifically cytoplasmic gelsolin (cGSN), is an abundant secretory protein. The exported isoform of gelsolin has additional 25 amino acids, derived from alternative splicing of a single gene.

Recombinant human gelsolin (rhGSN) (Biogen DEC, Inc., Cambridge, Mass.) is produced in Escherichia coli (E. coli) and has the same primary structure as a natural protein. However, due to a disulfide bond present in a natural protein under standard purification conditions, the rhGSN is different from natural human plasma gelsolin. Accordingly, the recombinant protein is completely oxidized after purification, and its structure and functions are indistinguishable from human plasma gelsolin (Wen et al., Biochemistry. 35:9700, 1996). In some of the important therapeutic aspects and embodiments of the present invention, the use of rhGSN is preferred. In some of the important diagnostic aspects and embodiments of the present invention, the use of plasma gelsolin (pGSN) is preferred.

In addition, there is provided a method of inducing differentiation to a dendritic cell, the method including treating gelsolin with immature dendritic to induce differentiation of the immature to mature dendritic cells.

In detail, the immature dendritic cell is obtained by treating interleukin-4 (IL-4) and granulocyte-macrophage colony-stimulating factor (GM-CSF) with a bone marrow-derived hematopoietic stem cell. Preferably, the bone marrow-derived hematopoietic stem cell may be treated 3 times every 2 days with GM-CSF a recombinant mouse in a concentration of 10 to 20 ng/ml and IL-4 in a concentration of 5 to 10 ng/ml.

Preferably, gelsolin may be treated in a range of about 0.1 to 5 ug/ml for 12 to 48 hours.

In addition, there is provided a dendritic cell differentiated according to the method of inducing differentiation. In detail, the dendritic cell is characterized by an increase in secretion of IL-12p70 and IL-23 cytokines an increase in proliferation of a T lymphocyte, and an increase in secretion of IFN-γ and IL-4 cytokines.

In addition, there is provided a vaccine composition including the dendritic cell as an effective ingredient for treating cancer or other immune-mediated diseases.

The term “cancer” as used herein refers to breast cancer, but is not limited thereto.

The term “immune-mediated disease” as used herein refers to an immune disease where a Th2 cell is activated, and examples of the immune disease are asthma and atopy, but are not limited thereto.

When the vaccine composition of the present invention is prepared in an injectable formulation, the vaccine composition is generally prepared in a form of sterile aqueous solution or dispersion solution. In order to prevent contamination by microorganisms, the vaccine composition of the present invention may further include an antimicrobial agent or a stabilizer. Appropriate examples of the antimicrobial agent are paraben, chlorobutanol, phenol, sorbic acid, and gentamycin. Appropriate examples of the stabilizer are carbohydrates, such as glycerol/EDTA and sorbitol, proteins, such as albumin and casein, and gelatin.

The vaccine composition of the present invention may be prepared in a form of a buffer solution according to methods known in the art using sodium phosphate, sodium dihydrogen phosphate, potassium phosphate, or potassium diydrogen phosphate. The vaccine composition of the present invention may be orally or parenterally administered. In the case of parenteral administration, subcutaneous, intramuscular, intravenous, or intraarterial administration methods may be used. Meanwhile, dosage of the vaccine composition of the present invention may be determined according to a dosage schedule, a unit dosage of a fusion antibody included in formulations, and a patient's health condition.

In order to enhance immunogenicity of the vaccine composition, an immunostimulant, such as cytokine, cholera toxin, or salmonella toxin, may be added to the composition. In addition, the vaccine composition may be combined with an adjuvant, such as alum, incomplete Freund's adjuvant, MF59 (oil emulsion), MTP-PE (i.e., a derivative of the mycobacterial cell wall component MDP), and QS-21 (derived from soapbark tree Quilaja saponaria).

In addition, in order to enhance efficiency of the vaccine composition based on the dendritic cell, when injecting the dendritic cell, the dendritic cell may be injected in combination with cytokines, such as IL-12 that helps T cell activation. Also, a dendritic cell to which a cytokine gene is transfected may be used.

Cells including a dendritic cell as an effective ingredient of the vaccine composition of the present invention are vaccinated as a therapeutic vaccine for the human body, and thus, the cell proliferation may be suppressed to increase stability of the vaccine. For example, in consideration of safe use of cell vaccines, cells may be optionally treated by heating, radiation, or mitomycin C (MMC), and accordingly, proliferation of the cells may be suppressed without affecting the vaccine function. For example, in the case of X-ray radiation treatment, a total radiation amount of about 1000 to 3300 Rads may be used. In the case of MMC treatment, 25˜50 μg/μl of MMC are added to a dendritic cell, and then, the mixture is heat-retained at a temperature of 37° C. for about 30 to 60 minutes. In the case of heat treatment, the cells may be heated at a temperature of 50˜65° C. for 20 minutes.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 shows images showing a shape of immature dendritic cells after 6 days of treatment with IL-4 and GM-CSF in which the dendritic cells are extracted from mouse bone marrow;

FIG. 2 is a view showing results of analyzing cell surface phenotypes regarding differentiation to mature dendritic cells as shown on representative differentiation-inducing factors, e.g., LPS or IFNg/TNFα, or gelsolin;

FIG. 3 is a view showing results in comparison with a control group in a percentage regarding phenotypic expression of cells in a group of total dendritic cells;

FIG. 4 is a view showing results in comparison with a control group regarding mean fluorescence intensity in each of the phenotype expressed on the cell surface;

FIG. 5 is a view showing results of measuring an amount of cytokine produced in a dendritic cell by gelsolin;

FIG. 6 shows microscope images resulted from co-cultivation of dendritic cells and allogeneic mixed leukocytes in which the dendritic cells are matured by differentiation-inducing factors, e.g., LPS or IFNg/TNFα, or gelsolin;

FIG. 7 is a view showing results of proliferation of allogeneic mixed leukocytes with the dendritic cell treated as in FIG. 6;

FIG. 8 is a view showing results of measuring cytokine secreted from allogeneic mixed leukocytes with dendritic cells, wherein the proliferation of the leukocytes is induced by the dendritic cells that are matured by the same treatment as in FIG. 6;

FIG. 9 is a view showing results of intracellular cytokines expression of leukocytes that are differentiated by dendritic cells that matured by differentiation-inducing factors, e.g., LPS or IFNg/TNFα, or gelsolin;

FIG. 10 is a view showing results in comparison with a control group in a percentage regarding intracellular cytokine expression in leukocytes that are differentiated by a group of total dendritic cells;

FIG. 11 is a view showing results of analyzing surface phenotype expressed on the cell surface of dendritic cells that are loaded or not loaded with a mouse breast cancer antigen;

FIG. 12 is a view showing results in comparison with a control group in a percentage regarding cells having phenotype expression in a group of dendritic cells that are treated the same as in FIG. 11;

FIG. 13 is a view showing results of measuring an amount of cytokine IL-12p70 secreted from dendritic cells that are loaded or unloaded with a mouse breast cancer antigen;

FIG. 14 is a view showing results of measuring an amount of cytokine IL-23 produced in dendritic cells that are loaded or unloaded with a mouse breast cancer antigen;

FIG. 15 is a view showing results of intracellular signaling mechanism that is activated by gelsolin in a dendritic cell.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, the present inventive concept will be described in further detail with reference to the following examples. These examples are for illustrative purposes only and are not intended to limit the scope of the present invention.

### Example 1

### Preparation of Dendritic Cells Derived from Mouse Bone Marrow

6-week-old C57BL/6 and BALB/c mice were purchased from Narabiotech (South Korea). After an acclimation period of 1 week in the laboratory animal facility of Korea Institute of Radiational and Medical Sciences, the mice were used for the experiments. All the procedures carried out herein were followed according to the Guide for the Care and Use of Laboratory Animals, Korea Institute of Radiational and Medical Sciences.

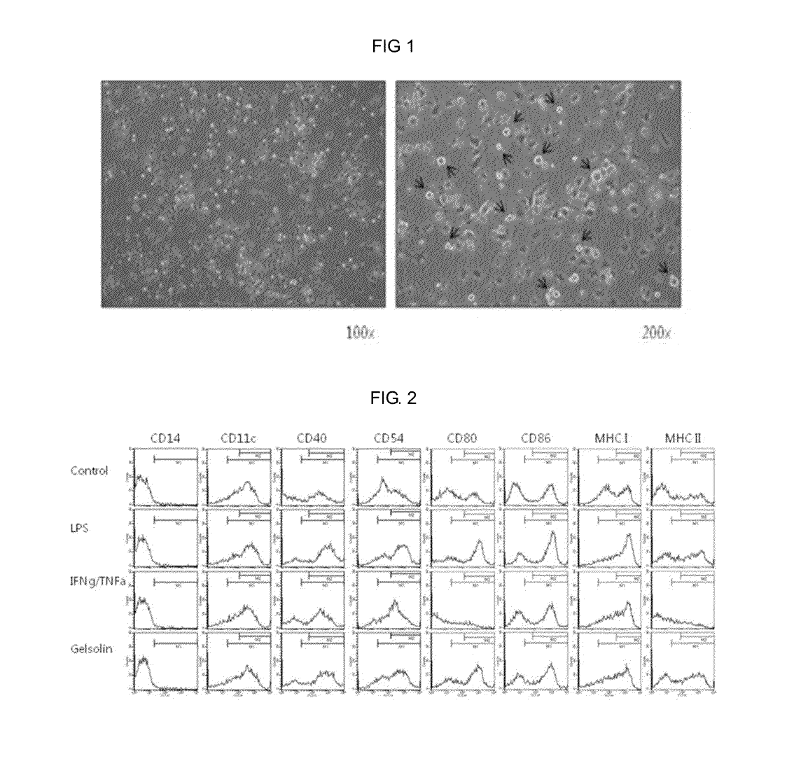

Dendritic cells obtained from mouse bone-marrow were used. In order to secure a large amount of the number of the dendritic cells, Inaba's culture method (Inaba et al., J. Exp. Med. 175:1157, 1992), which is a representative method of culturing a dendritic cell, was modified and applied to the dendritic cells. The number of the dendritic cells obtained from bone-marrow cells was at about 10 to 15% of bone marrow cells in an initial culture. A tibia and a femur of a mouse aged 6 to 11 weeks were extracted, and a RPMI-1640 medium was added to bone marrow of the tibia and the femur to obtain bone marrow cells therefrom. Then, hypotonic ammonium chloride-potassium (ACK) lysing buffer was used for the lysis of red blood cells. The bone marrow cells were cultured in an RPMI-1640 medium containing about 10 to 20 ng/ml recombinant mouse GM-CSF, about 5 to 10 ng/ml recombinant mouse IL-4, 5 to 10% heat-inactivated fetal bovine serum, L-glutamine, 25 mM of HEPES, penicillin, and streptomycin under conditions of 5% CO₂ and a temperature of 37° C., thereby inducing differentiation into dendritic cells. The medium was replaced every 2 days with a medium containing the recombinant cytokines. After 2 and 4 days of the culture, granulocytes and lymphocytes that were floating on the culture dish were removed. After 6 days of the culture, as shown in FIG. 1, dendritic cells that were differentiated from precursor cells attached on the bottom of the culture dish and that had characteristic protrusions in suspension were used in the experiments. In order to confirm purity of the prepared dendritic cells, the dendritic cells were stained with FITC-conjugated anti-CD11c antibody against CD11c molecule that is highly expressed on a surface. Then, it was confirmed by flow cytometry that the ratio of the CD11c-positive dendritic cells was 85% or more.

### Example 2

### Identification of Maturity of Dendritic Cells Upon Recombinant Gelsolin Protein Based on Surface Phenotype of Dendritic Cells

Recombinant human gelsolin protein (MyBioSource, USA) was purchased and applied to the dendritic cells of Example 1. Then, differentiation into dendritic cells and changes in functions of the dendritic cells were deduced by using flow cytometry based on the expression of surface molecules of the dendritic cells. Maturation of the dendritic cells was a part of steps of the differentiation, referring to a process of acquiring capability that APCs had. During maturation, the dendritic cells underwent a transformational change, an increase in the expression of costimulatory molecules, adhesion molecules, and MHC molecules on the cell surface, and an increase in transportation to lymph nodes. Accordingly, antigens were exposed to unsensitized lymphocytes so as to induce proliferation and differentiation of the lymphocytes.

About 1,000,000 to 2,000,000 of the dendritic cells prepared above were subjected to flotation in about 2 to 4 ml of the medium prepared above to be placed in a 6-well plate. Then, the dendritic cells were treated with recombinant human gelsolin in a concentration in a range of about 0.1 to 5 ug/ml. As a positive control group, the dendritic cells were treated with endotoxin (LPS, E. coli serotype O55:B5) in a range of about 0.01 to 1 mg/ml, which is one of representative materials that induce maturation of the dendritic cells, or with IFN-g in a range of about 10 to 100 ng/ml and TNF-α in a range of about 10 to 50 ng/ml. After 12 to 48 hours of the culture, a fluorescent material-labeled antibody with respect to surface molecules of the dendritic cells (e.g., anti-CD11c-PE, anti-CD54-FITC, anti-CD4O-PE, anti-CD80-PE, anti-CD86-FITC, anti-H-2K[b] or H-2K[d]-PE, or anti-I-A[b] or I-A[d]-FITC) were used to stain the dendritic cells for 20 minutes to 1 hour at a temperature of 4° C. Then, the results were obtained by flow cytometry. As shown in FIGS. 2, 3, and 4, it was observed that the amount of maturation markers on the surface of the dendritic cells was increased by gelsolin treatment. That is, the increased expression of CD40, CD80, CD86, and MHC class II in the gelsolin-treated dendritic cells was found to be the same with or greater than those in the dendritic cells in a positive control group treated with LPS or IFNg/TNFα.

### Example 3

### Expression of Cytokine of Dendritic Cells Upon Recombinant Gelsolin Protein

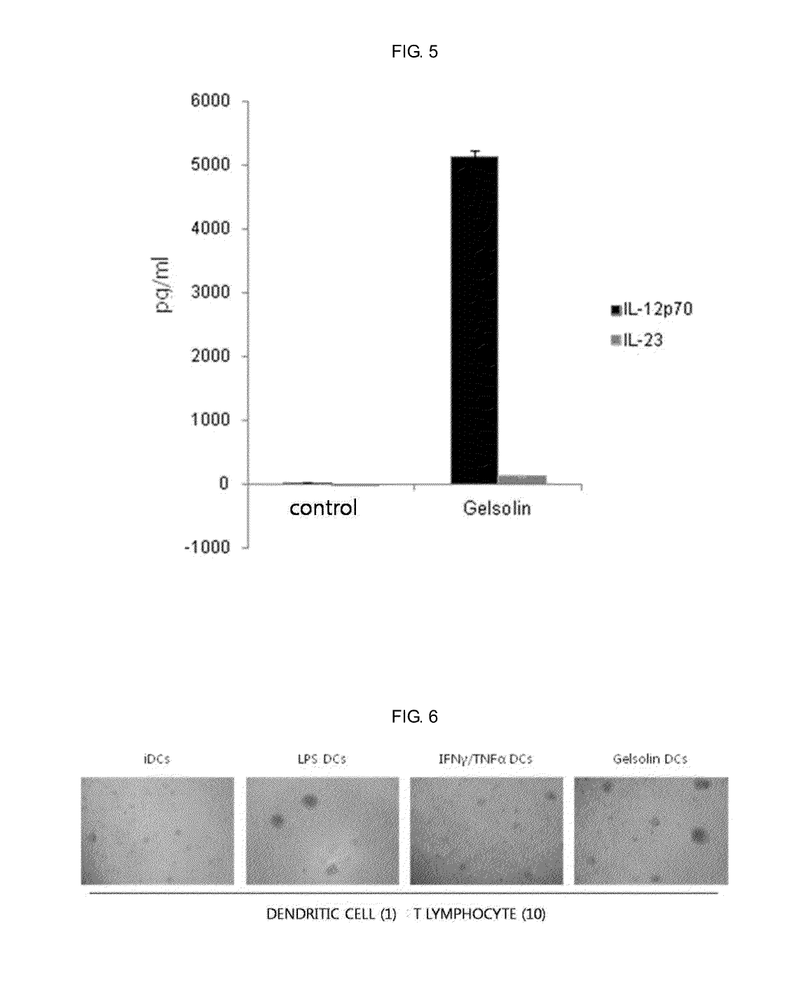

In order to measure the amounts of cytokine secreted by gelsolin from the dendritic cells and contributing to proliferation and inhibition of T lymphocytes, sandwich ELISA was carried out. The dendritic cells were treated with gelsolin under the same conditions of Example 2, and then, the amounts of IL-12p70 were measured, wherein IL-12p70 was made of p40 and p35 subunits of IL-12 that was representative inflammatory cytokine secreted from activated dendritic cells and contributed to proliferation and differentiation of T lymphocytes to play an essential role in the induction of cellular immune response. In addition to IL-12p70, the amounts of cytokine IL-23 were also measured, wherein IL-23 belonging to the IL-12 family was made of p40 and p19 subunits and had an important role in the development of Th17 cells. As shown in FIG. 5, the secretion of IL-12p70 and IL-23 increased in the gelsolin-treated dendritic cells.

### Example 4

### Proliferative Response of Dendritic Cells and Allogeneic Mixed Leukocytes Upon Recombinant Gelsolin Protein

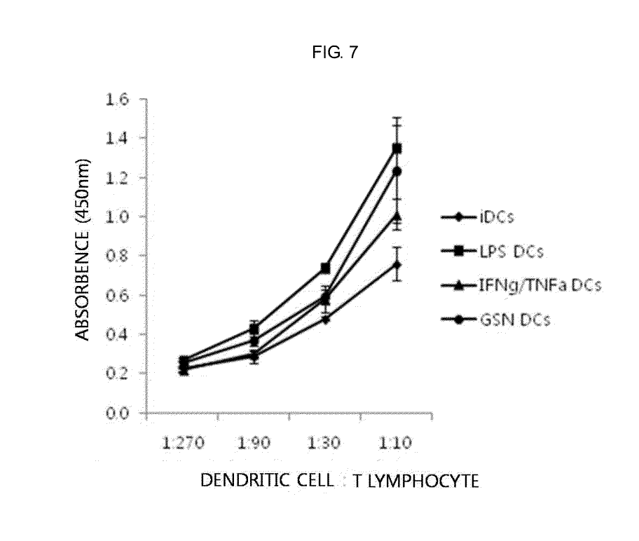

An allogeneic mixed leukocyte reaction (MLR) is a method typically used to measure activation of co-stimulatory molecules, and in this regard, the MLR is also considered to have standard techniques for evaluating function of the dendritic cells as APCs. The dendritic cells that were treated with LPS or gelsolin under the same conditions of Example 2 and cultured for 24 hours were collected, and then, the medium was washed out twice to be cultured with spleen-derived T lymphocytes of an allogeneic mouse in a 96-well plate (100,000-400,000 cells/well) at a ratio of 1:270, 1:90, 1:30, and 1:10. The T lymphocytes were cultured in an RPMI-1640 containing 10-20% FBS, L-glutamine, HEPES, penicillin, streptomycin, 0.1 mM non-essential amino acid, and 1 mM sodium pyruvate. After 4 days of the culture, each well of plate was treated with 10 to 20 ul of a cell-counting kit-8 (CCK-8) solution (Dojindo, Japan). After 4 hours of the reaction, absorbance of the cells was measured at a wavelength of 450 nm. When the dendritic cells and the T lymphocytes were co-cultured, a number of aggregates was formed, and that is, these aggregates denote allospecific T lymphoblast clusters stimulated by the dendritic cells. As shown in FIG. 6, in the case of co-culture of the gelsolin-treated dendritic cells and the T lymphocytes, a number of large aggregates was formed in comparison with a positive control group, i.e., a group of the dendritic cells treated with LPS or IFNγ/TNFα. As shown in FIG. 7, the proliferation of the T lymphocytes upon CCK-8 was in a similar manner with that of a positive control group, i.e., a group of the LPS-treated dendritic cells, whereas the proliferation of the T lymphocytes upon CCK-8 was significantly increased than that of a positive control group, i.e., a group of the IFNγ/TNFα-treated dendritic cells.

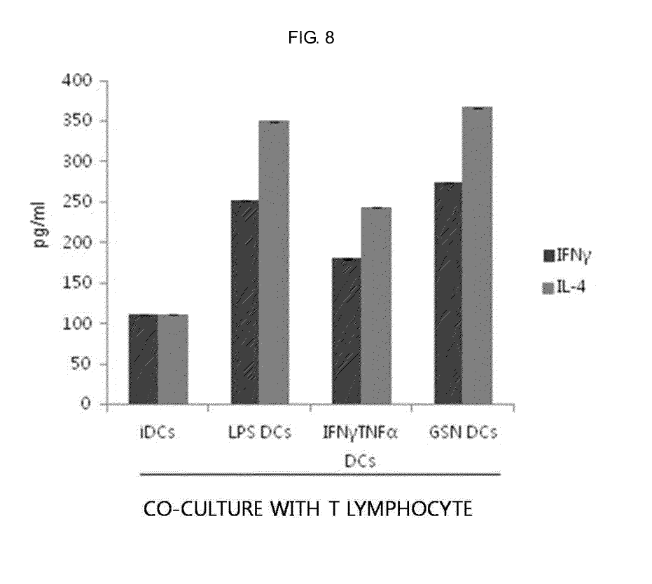

In order to identify the direction of differentiation in T lymphocytes that were activated by the dendritic cells, an allogeneic mixed lymphocyte reaction was carried out (at a ratio of 1:10) under the same conditions of Example 4 to quantify IFN-γ and IL-4, which were representative cytokines with respect to a reaction of Th1/Th2 secreted in cell culture. As shown in FIG. 8, it was confirmed that the secretion of IFN-γ and IL-4 significantly increased as in a positive control group by the gelsolin treatment.

### Example 5

### Confirmation of Differentiation of T Lymphocytes of Dendritic Cells Upon Recombinant Gelsolin Protein

It was confirmed that the gelsolin treatment increased proliferation of T lymphocytes and secretion of IFN-γ and IL-4 based on ELISA analysis. In the present Example, BD Cytofix/Cytoperm Fixation/Permeabilization Kit (BD Bioscience, USA) was used to perform immunofluorescence on dendritic cells by using a surface marker of T lymphocyte and an antibody against intracellular cytokine. According to flow cytometry, the ratio of cells secreting cytokines (e.g., IFN-γ, IL-4, or IL-17A) that are specific to CD4-positive T lymphocytes was measured, thereby re-confirming the direction of differentiation of activated T lymphocytes (e.g., Th 1, Th 2, or Th 17 cells). The dendritic cells were treated with LPS under the same conditions of Example 4, and then, co-cultured with spleen cells of an allogeneic mouse at a ratio of 1:10 in the medium prepared above for 72 to 120 hours. In order to maintain the produced cytokines within the cells without being secreted out of the cells, the cells were treated with BD GolgiStop™ reagent for 1 to 6 hours according to methods provided herein, the reagent containing intracellular protein transport inhibitor, monensin. Afterwards, the cells were collected. In order to reduce non-specific immunostaining through Fc receptors present on the cell surface, 1 to 2 ug of BD Fc Block™ (Bioscience) was used for a reaction at a temperature of 4° C. for 15 to 30 minutes, and then, an FITC-conjugated anti-CD4 antibody (BD, Bioscience) was used to stain the cell surface at a temperature of 4° C. for 20 minutes to 1 hour. Then, the cells were washed out twice by PBS containing 1-5% fetal bovine serum and 0.05-0.1% sodium azide. Before staining the intracellular cytokines, a fixation/permeabilization solution previously provided was used for a reaction at a temperature of 4° C. for 20 minutes to 18 hours. Also, a BD Perm/Wash™ reagent previously provided was used to wash out the cells twice, and then, anti-IFN-γ, anti-IL-4, and anti-IL-17A antibodies conjugated with PE were used to perform immunostaining. The results were obtained by flow cytometry.

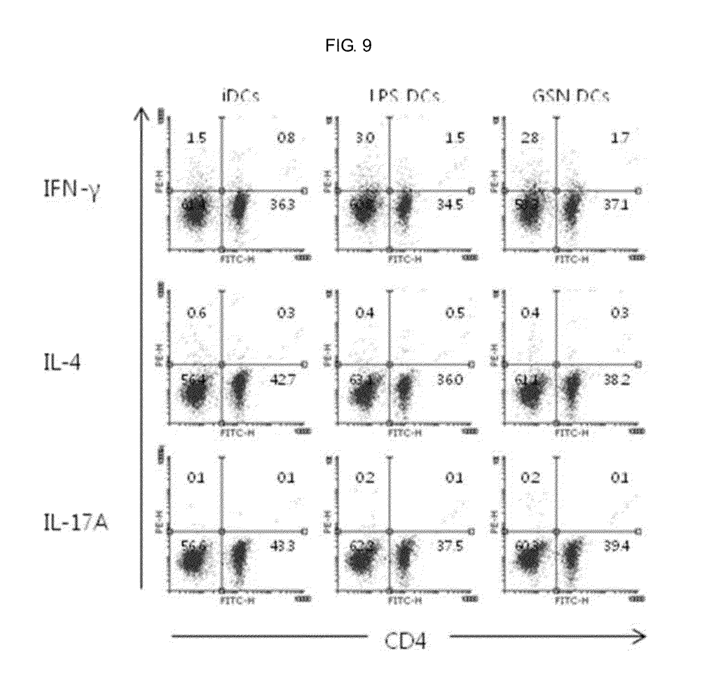

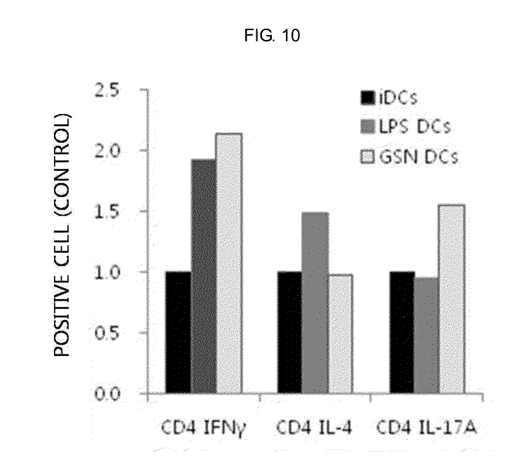

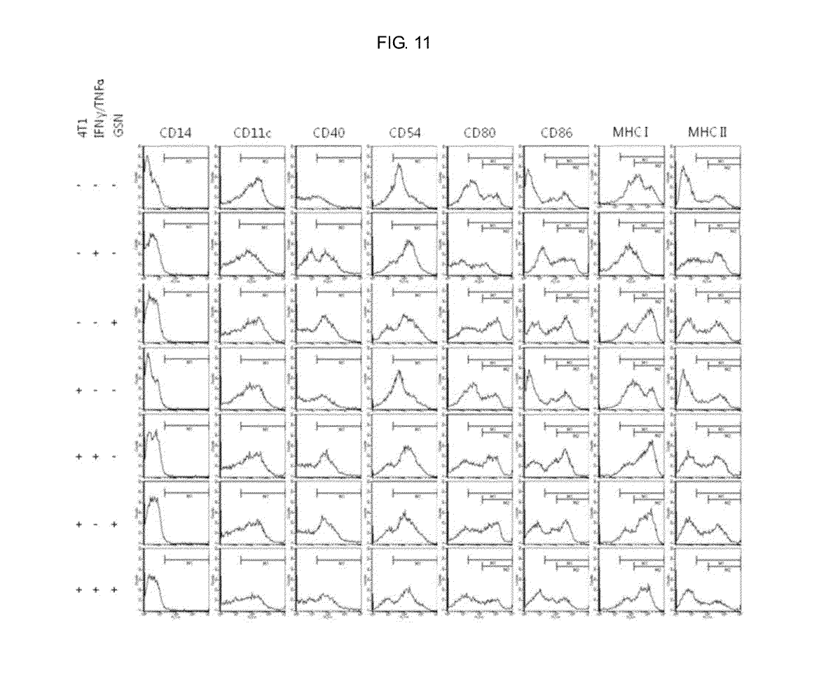

As shown in FIGS. 9 and 10, it was confirmed that the ratio of cells that produced CD4-positive IFN-γ was increased in the gelsolin-treated group as much as the LPS-treated group. Also, it was confirmed that the ratio of cells that produced CD4-positive IL-17A was significantly increased in the gelsolin-treated group as compared with that in the LPS-treated group.

That is, it is deemed that the dendritic cells matured by the gelsolin protein increased the Th1 reaction in the T lymphocytes.

### Example 6

### Changes in Surface Phenotype of Dendritic Cells Loaded with Breast Tumor Antigens of Mouse

Before identifying anticancer activity of the matured dendritic cells, the dendritic cells were loaded with tumor antigens and treated with gelsolin so as to observe changes in surface phenotype of the dendritic cells.

400,000 to 500,000 of mouse breast cancer cells (4T1 cell line, CRL-2539, ATCC, USA) were contained in a 100 mm culture dish and cultured in an RPMI-1640 medium containing 5 to 10% heat-inactivated fetal bovine serum, L-glutamine, penicillin, and streptomycin under conditions of 5% CO₂ and a temperature of 37° C. After 24 to 72 hours of the culture, the medium was treated with a trypsin-EDTA solution to collect the dendritic cells. In order to load the dendritic cells with tumor antigens, a suspension solution was prepared so as to contain 10,000,000 to 20,000,000 of the 4T1 cells per 1 ml of the same medium, and then, the suspension solution was subjected to repeated freeze-thaw cycles at liquid nitrogen and a temperature of 37° C., thereby inducing apoptosis of the tumor cells. Afterwards, the centrifugation was performed at a speed of 13,000 rpm for 20 minutes, and a supernatant obtained therefrom was filtered by using a 0.2 um filter. The concentration of tumor lysate was measure according to the Bradford method of protein quantification. In preparation method of tumor lysate, methods such as high-dose ionizing radiation, anticancer drug treatment, or the like are already widely known in the art in addition to repeated freeze-thaw cycles. It is also reported that tumor-specific proteins are discharged and serve as antigens through the deactivation process of tumor cells (Yu et al., Cancer Res. 64:4973, 2004; Teitz-Tennenbaum et al., J. Immunother. 31:345, 2008; Kim et al., Cancer Letters. 335:278, 2013).

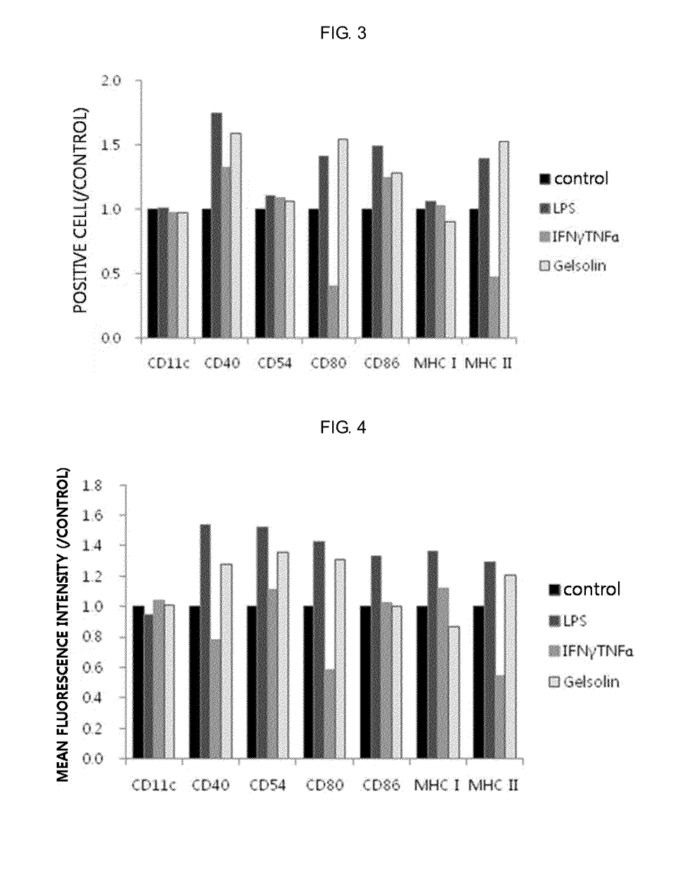

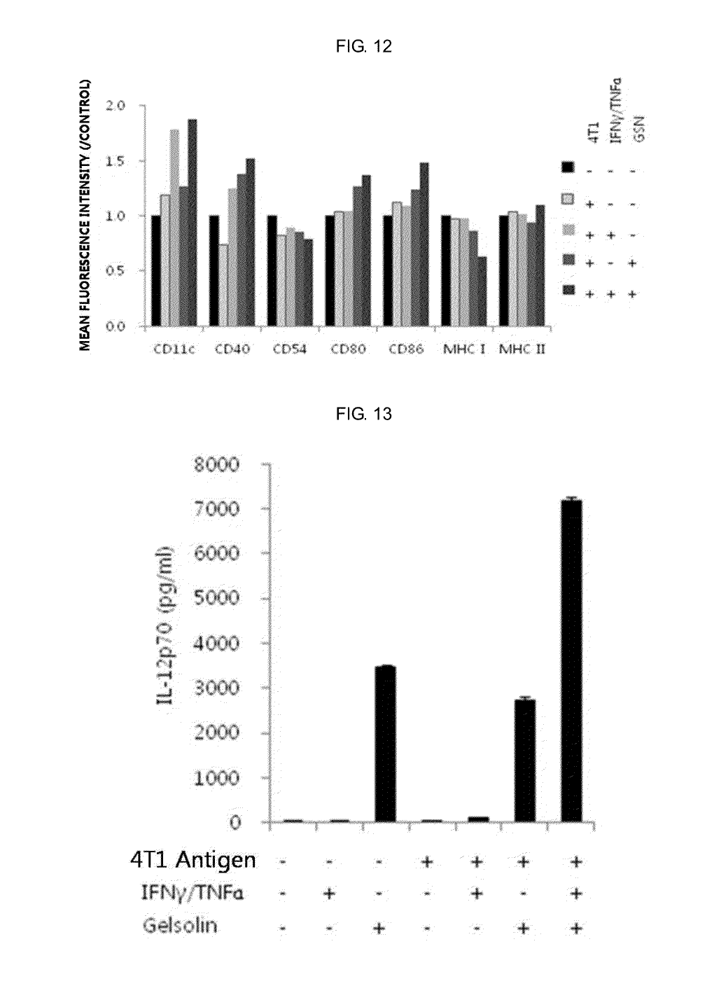

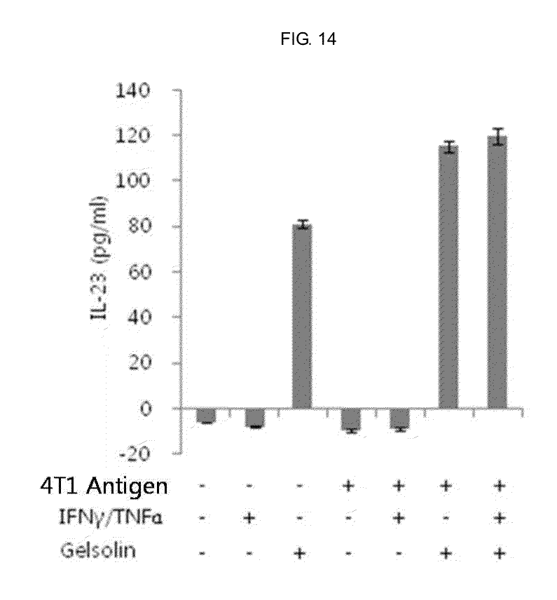

The dendritic cells prepared as described in Example 1 were co-cultured with 100 ug of the lysate of the mouse breast tumor cell lines. After 1 hour of the co-culture, under the same conditions described above, the dendritic cells were treated with recombinant mouse IFNγ/TNFα or gelsolin and then cultured again. Then, changes in surface phenotype of the dendritic cells were observed according to the methods described in Example 2. As shown in FIG. 11, the single treatment with IFNγ/TNFα or the single treatment with gelsolin significantly increased the expression of CD40, CD54, CD86, and MHC class II, thereby inducing maturation of the dendritic cells. The dendritic cells stimulated by the lysate of the mouse breast tumor cell lines were not significantly matured in the present Example. However, when treated in combination with IFNγ/TNFα or gelsolin, as shown in FIG. 12, the expression of CD40, CD80, and CD86 was increased, thereby inducing maturation of the dendritic cells. In addition, as shown in FIG. 3, the product amount of IL-12 was significantly increased in the dendritic cells treated with gelsolin only. When the dendritic cells were treated in combination with IFNγ/TNFα and the lysate of the mouse breast tumor cell lines, as shown in FIG. 13, the product amount of IL-12 was increased at least twice as large as the product amount of IL-12 in the dendritic cells treated with gelsolin only. FIG. 14 shows the results of measuring the product amount of IL-23. As shown in FIG. 13, it was confirmed that the product amount of cytokine was significantly increased in the dendritic cells that were treated with gelsolin only, and in addition, the product amount of cytokine in the dendritic cells that were treated in combination as described above was increased at least 1.5 times as large as that in the dendritic cells that were treated with gelsolin only.

### Example 7

### Mechanism of Activating Intracellular Signaling of Dendritic Cells by Gelsolin

As described above, the treatment of the dendritic cells with gelsolin induced maturation of the dendritic cells at a similar degree of maturation of the positive control group, i.e., the LPS-treated dendritic cells. In addition, the treatment of the dendritic cells with gelsolin showed immune activation and anti-tumor function, and accordingly, the representative signaling mechanism of the gelsolin-treated dendritic cells was identified. LPS is reported as an agonist of toll-like receptor (TLR) 4, and in this regard, the expression of the TLR 4, which is one of pattern-recognition receptors (PRRs), was identified by western-blotting analysis in the gelsolin-treated dendritic cells. Then, the expression of NF-κB, representing downstream signaling mechanism of the TLR4, and the expression of C-type mannose receptor (MRC2), which is one of C-type lectin receptors (CLRs) that is expressed on the surface of dendritic cells and macrophages and involved in phagocytosis of various glycoproteins, were identified. The dendritic cells that were treated with gelsolin and cultured for 24 hours under the same conditions described above were collected, and then, dissolved in an RIPA buffer solution (50 mM Tris-Cl (pH7.4), 1% NP-40, 150 mM NaCl, 1 mM EDTA) containing protease inhibitor (1 mM PMSF, 1 ug/ml aprotinin, 1 ug/ml leupeptin, and 1 mM Na₃VO₄) so as to prepare protein lysate. Afterwards, 4 to 20% SDS-PAGE was performed thereto to separate proteins. The proteins separated on the SDS-PAGE gel were transferred to a nitrocellulose membrane (BioRad, Hercules, Calif., USA), and the membrane was subjected to blocking by 5 to 10% non-fat milk for 1 hour at room temperature. Then, anti-TLR4 antibody (Santa cruz, USA), anti-MRC2 antibody (Santa cruz, USA), anti-NF-κB antibody (Santa cruz, USA), and anti-beta-actin antibody (Sigma, USA) were used for a reaction on the membrane for 16 to 48 hours at a temperature of 4° C. Afterwards, the membrane was horseradish peroxidase (HRP)-conjugated secondary antibody was used for a reaction on the membrane at room temperature for 1 hour, and then, the results were visualized by using the ECL kit (Amersham, United Kingdom).

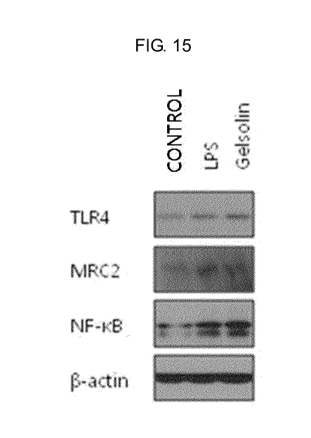

As shown in FIG. 15, it was observed that the expression of TLR4, MRC2, and NF-κB was increased in the gelsolin-treated dendritic cells in a similar manner as in the LPS-treated dendritic cells.

As described above, according to the one or more of the above exemplary embodiments, provided are a composition including gelsolin as an effective ingredient for inducing differentiation into dendritic cells, a method of inducing differentiation by using the composition, and a vaccine composition for treating cancer or an immune disease by using the method. In detail, treatment using gelsolin catalyzes differentiation into matured dendritic cells, and the composition disclosed herein shows simple and excellent immunity enhancement effects as compared with the existing differentiation composition factors. In this regard, the composition disclosed herein can be preferably used for preparing various vaccines for the dendritic cells, and for example, may be applied to the treatment of diseases requiring Th1 immune cell environment as in the treatment of cancer, asthma, and atopy.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of inducing differentiation into a dendritic cell, comprising:
providing a composition comprising gelsolin as an active ingredient.

2. The method of claim 1, wherein the dendritic cell comprises at least one positive immunological characteristic on a surface antigen selected from the group consisting of CD40, CD80, CD86, and MHC class II molecules.

3. A method of inducing differentiation into a dendritic cell, the method comprising:
treating an immature dendritic cell with gelsolin.

4. The method of claim 3, wherein the immature dendritic cell is obtained by treating a bone marrow-derived hematopoietic stem cell with interleukin-4 (IL-4) and a granulocyte-macrophage colony-stimulating factor (GM-CSF).

5. The method of claim 3, wherein the gelsolin is treated in a concentration of 0.1 to 5 μg/ml for 12 to 48 hours.

6. A dendritic cell differentiated by the method of claim 3.

7. The dendritic cell of claim 6, wherein the dendritic cell is characterized by an increased amount of cytokine, such as IL-12p70 and IL-23.

8. The dendritic cell of claim 6, wherein the dendritic cell increases proliferation of a T lymphocyte and secretion of cytokine, such as IFN-γ and IL-4.

9. A vaccine composition including the dendritic cell of claim 6 as an effective ingredient for treating cancer or an immune-mediated disease.

10. The vaccine composition of claim 9, wherein a type of the cancer is breast cancer.

11. The vaccine composition of claim 9, wherein the immune-mediated disease is asthma or atopy.
